(19) United States
(12) Patent Application Publication
(10) Pub. No.: US 2018/0256381 A1
(43) Pub. Date: Sep. 13, 2018

(54) KNEE RETRACTOR

(71) Applicants: Ki Yong CHANG, Seoul (KR); Min Young KANG, Seoul (KR)

(72) Inventor: Ki Yong CHANG, Seoul (KR)

(21) Appl. No.: 15/743,690

(22) PCT Filed: Jul. 11, 2016
(86) PCT No.: PCT/KR2016/007467

§ 371 Date: Jan. 11, 2018

(30) Foreign Application Priority Data
Aug. 7, 2015 (KR) 10-2015-0111611

(51) Int. Cl. A61F 5/048; A61F 5/01

(52) U.S. Cl. CPC A61F 5/048; A61H 3/00; A61F 5/0123

(57) ABSTRACT

A knee retractor according to an embodiment comprises: an upper frame, which is positioned on the thigh, and which has an upper retracting member provided on an end thereof; a lower frame, which is positioned on the lower leg region, and which has a lower retracting member provided on an end thereof so as to be able to engage with the upper retracting member and to rotate accordingly; and a band portion configured to surround the thigh and the lower leg region, the upper frame and the lower frame being mounted on the band portion, wherein contacting parts of the upper and lower retracting members can be positioned on the front side of the knee joint.

### TECHNICAL FIELD

The following description relates to a knee retractor, and more particularly, to a knee retractor in which contacting parts of an upper retracting member and a lower retracting member are located in front of a knee joint and that may simultaneously obtain a stable traction effect and a compression effect by a band portion.

### BACKGROUND ART

A joint generally refers to a point at which at least two bones forming a skeleton are connected, and all movements of a human body may be performed in joints. Joints enabling the above movements may be referred to as movable joints, and most of skeletons of a human body may be connected by movable joints.

A knee joint among various joints corresponds to a middle portion of a leg, and refers to a hinge-shaped joint between knee bones.

The knee joint may be involved in knee bending. Also, due to instability of a structure of bones of the knee joint, tissues other than joints, for example muscles, ligaments, joint membranes, and the like, may contribute to stability of the knee joint.

However, bones, ligaments, and the like may be injured due to a degenerative change or injury of cartilages that protect joints, which may lead to inflammation and pain. The inflammation and pain are called arthritis. When these symptoms persist, deformity of joints may be caused. In particular, degenerative arthritis of a knee may be a major cause of a chronic physical disorder.

Research has been conducted to treat the above arthritis, and Korean Patent Application No. 10-2010-0090619 filed in Jul. 13, 2009, discloses a knee brace detachably coupled to an ankle brace.

### DETAILED DESCRIPTION OF INVENTION

### Technical Subject

Example embodiments provide a knee retractor in which a frame having a traction function is coupled to a band portion that may be easily worn on a leg.

Example embodiments provide a knee retractor including a frame that is formed of a plastic material, and accordingly may be easily bent, may properly fit a shape of a leg, may enhance a wearing sensation and may be light in weight.

Example embodiments provide a knee retractor in which contacting parts of an upper retracting member and a lower retracting member are located in front of a knee joint, and accordingly may effectively support the knee joint.

Example embodiments provide a knee retractor that may be worn on a leg of a patient by a band portion, may effectively operate, and may press the leg.

Example embodiments provide a knee retractor that may securely fix a leg of a patient using a plurality of straps.

Example embodiments provide a knee retractor that may efficiently limit, using a stopper, an angle at which a knee joint is bent.

Example embodiments provide a knee retractor that may include a connection member that is located adjacent to a knee joint, and accordingly may reduce manufacturing costs and facilitate manufacturing.

Example embodiments provide a knee retractor that may protect cartilages by reducing load applied to a knee joint, may relieve pain of the knee joint, may induce healing of the knee joint, and may support the knee joint so that a user may naturally straighten and bend the knee joint.

### Technical Solutions

According to an aspect of the present invention, there is provided a knee retractor including: an upper frame located on a thigh region and including an upper retracting member located at an end; a lower frame located on a lower leg region and including a lower retracting member that is located at an end and that is rotatable in engagement with the upper retracting member; and a band portion provided to surround the thigh region and the lower leg region, the upper frame and the lower frame being fixed to the band portion, wherein contacting parts of the upper retracting member and the lower retracting member are located in front of a knee joint.

An end portion of each of the upper retracting member and the lower retracting member may have serrations so that the upper retracting member and the lower retracting member rotate in engagement with each other.

Each of the upper frame and the lower frame may have a “Y” shape. The upper frame and the lower frame may include first portions extending from the knee joint in a longitudinal direction of each of the thigh region and the lower leg region; and second portions connected to the first portions and provided to surround an upper portion and a lower portion of the knee joint.

The knee retractor may further include upper side frames located on both sides of the thigh region, and each having one end fixed coaxially with the upper retracting member; and lower side frames located on both sides of the lower leg region, and each having one end fixed coaxially with the lower retracting member.

The upper side frames may each include an upper side straight member extending from a side of the thigh region, and an upper side diagonal member extending from the upper side straight member toward the upper retracting member. The lower side frames may each include a lower side straight member extending from a side of the lower leg region, and a lower side diagonal member extending from the lower side straight member toward the lower retracting member.

The upper side frame may rotate in conjunction with a rotation of the upper retracting member, and the lower side frame may rotate in conjunction with a rotation of the lower retracting member.

The band portion may include a plurality of straps. The plurality of straps may include a first strap passing through the upper frame and configured to fix the upper frame to the band portion; a second strap spaced downwardly from the first strap and configured to fix the upper side frame to the band portion; a third strap spaced downwardly from the second strap and configured to fix the lower side frame to the band portion; and a fourth strap spaced downwardly from the third strap, passing through the lower frame and configured to fix the lower frame to the band portion.

The knee retractor may further include a guide member in which a rotation axis of each of the upper retracting member, the lower retracting member, the upper side frame and the lower side frame is located.

According to another aspect of the present invention, there is provided a knee retractor including: an upper frame located on a thigh region and including an upper retracting member located at an end; a lower frame located on a lower leg region and including a lower retracting member that is located at an end and that is rotatable in engagement with the upper retracting member; upper side frames located on both sides of the thigh region, and each having one end fixed coaxially with the upper retracting member; and lower side frames located on both sides of the lower leg region, and each having one end fixed coaxially with the lower retracting member, wherein the upper side frame rotates in conjunction with a rotation of the upper retracting member, and the lower side frame rotates in conjunction with a rotation of the lower retracting member.

Contacting parts of the upper retracting member and the lower retracting member may be located in front of a knee joint.

The upper side frames may each include an upper side straight member extending from a side of the thigh region, and an upper side diagonal member extending from the upper side straight member toward the contacting parts of the upper retracting member and the lower retracting member. The lower side frames may each include a lower side straight member extending from a side of the lower leg region, and a lower side diagonal member extending from the lower side straight member toward the contacting parts of the upper retracting member and the lower retracting member.

The knee retractor may further include a band portion provided to surround the thigh region and the lower leg region. The upper frame, the lower frame, the upper side frame and the lower side frame may be fixed to the band portion.

The knee retractor may further include a guide member in which a rotation axis of each of the upper retracting member, the lower retracting member, the upper side frame and the lower side frame is located.

According to another aspect of the present invention, there is provided a knee retractor including: an upper side frame including an upper side straight member extending straightly from a side of a thigh region, and an upper side diagonal member extending diagonally from the upper side straight member toward an upper retracting member; and a lower side frame including a lower side straight member extending straightly from a side of a lower leg region, and a lower side diagonal member extending diagonally from the lower side straight member toward a lower retracting member, wherein a strength of a traction force is adjusted by adjusting a position of a rotation axis of each of the upper retracting member and the lower retracting member.

The strength of the traction force may increase as the position of the rotation axis of each of the upper frame and the lower frame is moved toward a front side of a knee joint, and a distance between an end portion of the thigh region and an end portion of the lower leg region may be increased by the traction force.

The upper retracting member and the lower retracting member may have circular shapes or quadrant shapes, and an end portion of each of the upper retracting member and the lower retracting member may have serrations so that the upper retracting member and the lower retracting member rotate in engagement with each other.

The knee retractor may further include a guide member in which a rotation axis of each of the upper retracting member, the lower retracting member, the upper side frame and the lower side frame is located. The guide member may be formed to extend from the upper side frame toward the lower side frame.

The guide member may include a first guide element located toward a lateral side of the knee joint; and a second guide element located toward the knee joint to face the first guide element. The first guide element may include a first hole and a second hole that are spaced apart from each other. The second guide element may include a first protrusion corresponding to the first hole and a second protrusion corresponding to the second hole. A hole may be formed in a central portion of the upper retracting member, and a hole may be formed adjacent to an end portion of the upper side frame. The hole of the upper retracting member and the hole of the upper side frame may be coupled to the first protrusion to overlap each other, and the upper frame and the upper side frame may rotate together about the first protrusion.

A hole may be formed in a central portion of the lower retracting member, and a hole may be formed adjacent to an end portion of the lower side frame. The hole of the lower retracting member and the hole of the lower side frame may be coupled to the second protrusion to overlap each other, and the lower frame and the lower side frame may rotate together about the second protrusion.

### Effects of Invention

According to example embodiments, by a knee retractor, a frame having a traction function may be coupled to a band portion that may be easily worn on a leg.

According to example embodiments, a knee retractor may include a frame that is formed of a plastic material, and accordingly may be easily bent, may properly fit a shape of a leg, may enhance a wearing sensation and may be light in weight.

According to example embodiments, in a knee retractor, contacting parts of an upper retracting member and a lower retracting member may be located in front of a knee joint, and accordingly it is possible to effectively support the knee joint.

According to example embodiments, a knee retractor may be worn on a leg of a patient by a band portion, may effectively operate, and may press the leg.

According to example embodiments, a knee retractor may securely fix a leg of a patient using a plurality of straps.

According to example embodiments, a knee retractor may efficiently limit, using a stopper, an angle at which a knee joint is bent.

According to example embodiments, in a knee retractor, a connection member may be located adjacent to a knee joint, and accordingly it is possible to reduce manufacturing costs and facilitate manufacturing.

According to example embodiments, by a knee retractor, it is possible to protect cartilages by reducing load applied to a knee joint, to relieve pain of the knee joint, to induce healing of the knee joint, and to support the knee joint so that a user may naturally straighten and bend the knee joint.

### BRIEF DESCRIPTION OF DRAWINGS

FIG. 1 illustrates a knee retractor according to an example embodiment.

FIG. 2 is an exploded view of a knee retractor according to an example embodiment.

FIGS. 3A and 3B illustrate a principle of providing a traction force in a knee retractor according to an example embodiment.

FIGS. 4A through 4C illustrate a change in a shape of a knee retractor according to an example embodiment when a knee is straightened.

FIG. 5 is a perspective view of a knee retractor according to an example embodiment.

FIG. 6 illustrates an example in which a cover is removed from a knee retractor according to an example embodiment and guide grooves have curved shapes.

FIG. 7 illustrates an example in which a cover is removed from a knee retractor according to an example embodiment and guide grooves have linear shapes.

FIGS. 8A through 8C illustrate examples of a stopper mounted in a knee retractor according to an example embodiment.

FIG. 9 illustrates an example in which a knee joint is straightened when a knee retractor according to an example embodiment is worn.

FIG. 10 illustrates an example in which a knee joint is bent when a knee retractor according to an example embodiment is worn.

### BEST MODE FOR CARRYING OUT THE INVENTION

Hereinafter, example embodiments will be described in detail with reference to the accompanying drawings. In the following description, the same elements will be designated by the same reference numerals although they are shown in different drawings. Further, in the following description of example embodiments, a detailed description of known functions and configurations incorporated herein will be omitted when it may make the subject matter of the example embodiments rather unclear.

Also, to describe components according to example embodiments, the terms first, second, A, B, (a), (b), etc. may be used herein. These terms are merely used to distinguish one component from another, but not to imply or suggest the substances, order or sequence of the components. It will be understood that when one component is described as being “connected,” “coupled,” or “linked” to another component, it may be directly connected or coupled to the other component, or intervening components may be present.

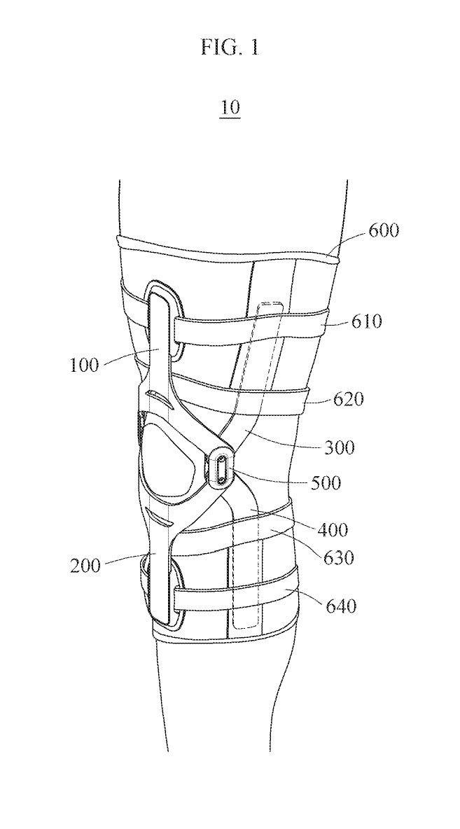

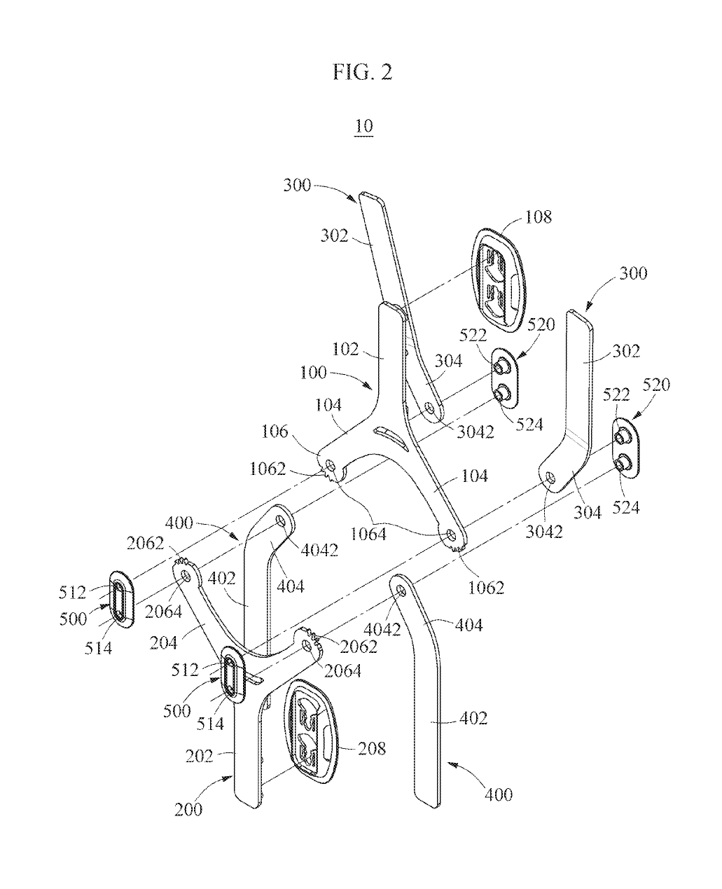

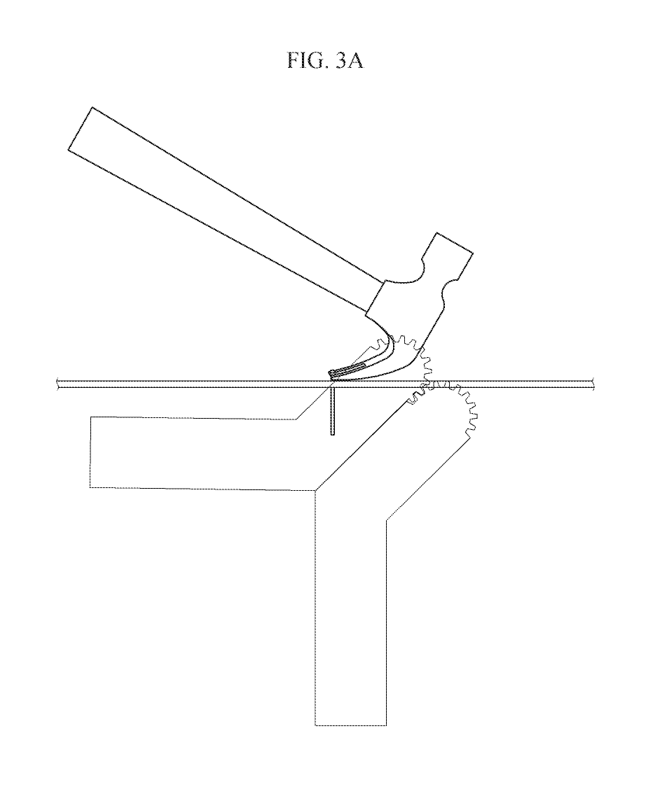

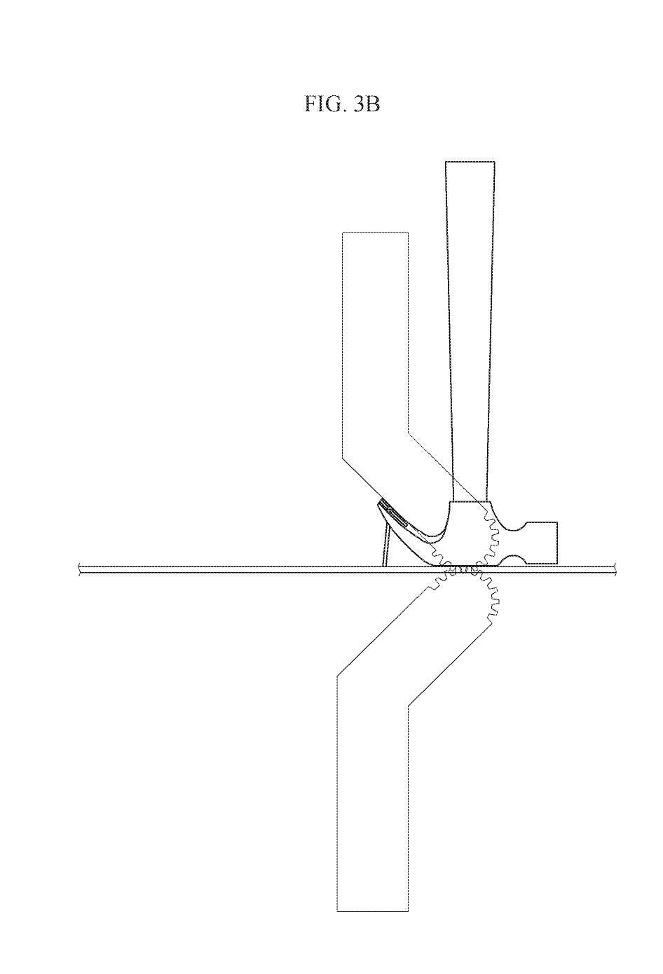

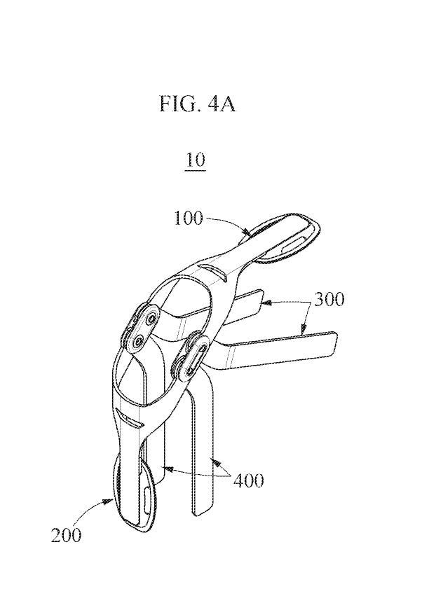

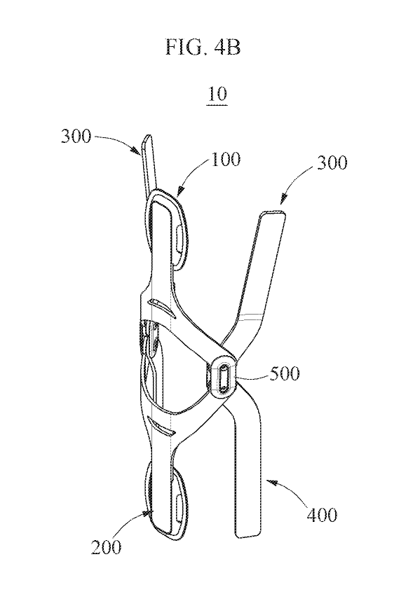

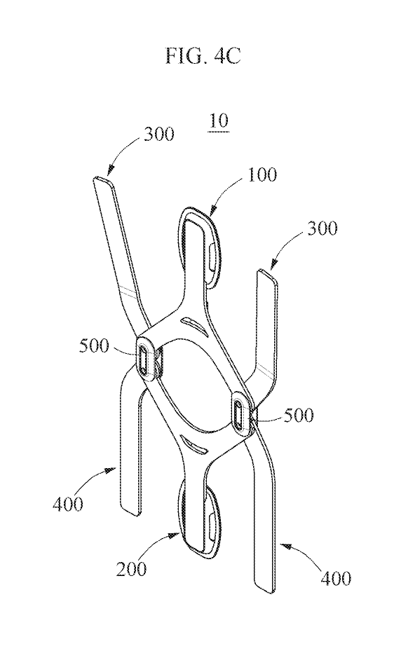

FIG. 1 illustrates a knee retractor according to an example embodiment, FIG. 2 is an exploded view of the knee retractor, FIGS. 3A and 3B illustrate a principle of providing a traction force in a knee retractor according to an example embodiment, and FIGS. 4A through 4C illustrate a change in a shape of the knee retractor when a knee is straightened. Referring to FIG. 1, a knee retractor 10 according to an example embodiment may include an upper frame 100, a lower frame 200, an upper side frame 300, a lower side frame 400, a guide member 500 and a band portion 600.

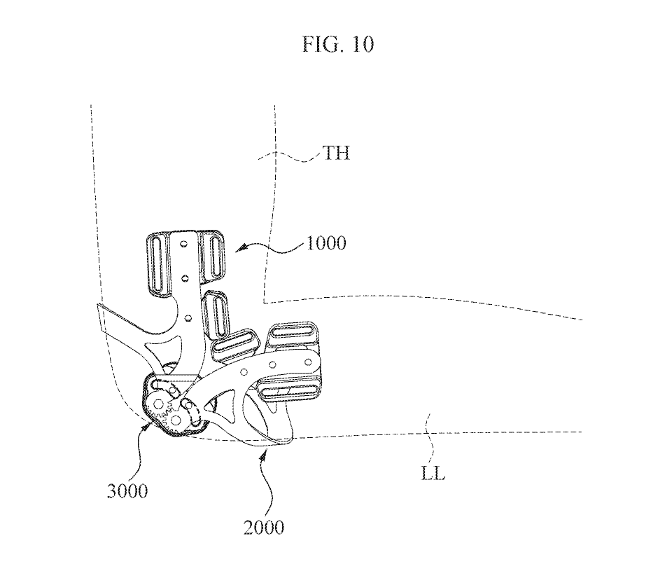

The upper frame 100 may be located in front of a thigh region (for example, a thigh region TH of FIG. 9 or 10).

The upper frame 100 may have a “Y” shape, as shown in FIG. 2.

For example, when the upper frame 100 has a “Y” shape, the upper frame 100 may include a first portion 102 and a second portion 104.

The first portion 102 may extend in a longitudinal direction of the thigh region from a knee joint in front of the thigh region.

The second portion 104 may be connected to the first portion 102 and may surround an upper portion of the knee joint. The second portion 104 may be formed to surround the thigh region in front of the thigh region.

Also, the upper frame 100 may include an upper retracting member 106 located at an end of the first portion 102 of the upper frame 100.

Since the upper frame 100 has the “Y” shape, two upper retracting members 106 may be provided. One of the upper retracting members 106 may be located in a medial side of a knee joint, and the other of the upper retracting members 106 may be located in a lateral side of the knee joint.

A portion of the upper retracting member 106 may have serrations 1062, and accordingly the upper retracting member 106 may rotate in engagement with serrations 2062 formed in a lower retracting member 206.

In addition, a hole 1064 may be formed in the upper retracting member 106, to fix the upper retracting member 106 to the guide member 500. For example, a protrusion 522 formed in the guide member 500 may be inserted into the hole 1064.

Also, the upper frame 100 may include a fixing element 108 located at an end of the first portion 102 of the upper frame 100.

The fixing element 108 may be formed integrally with or separately from the first portion 102.

For example, a strap, in particular, a first strap 610 may pass through the fixing element 108, and may fix the upper frame 100 to the band portion 600.

However, a shape or a structure of the fixing element 108 is not limited thereto, and the fixing element 108 may have any shape or structure to fix the upper frame 100 to the band portion 600.

Also, the lower frame 200 may be located in front of a lower leg region (for example, a lower leg region LL of FIG. 9 or 10) symmetrically to the upper frame 100.

The lower frame 200 may have a “Y” shape.

For example, when the lower frame 200 has a “Y” shape, the lower frame 200 may include a first portion 202 and a second portion 204.

The first portion 202 may extend in a longitudinal direction of the lower leg region from the knee joint in front of the lower leg region.

The second portion 204 may be connected to the first portion 202 and may surround a lower portion of the knee joint. The second portion 204 may be formed to surround the lower leg region in front of the lower leg region.

Also, the lower frame 200 may include a lower retracting member 206 located at an end of the first portion 202 of the lower frame 200.

Since the lower frame 200 has a “Y” shape, two lower retracting members 206 may be provided. One of the lower retracting members 206 may be located in the medial side of the knee joint and the other of the lower retracting members 206 may be located in the lateral side of the knee joint.

A portion of the lower retracting member 206 may have the serrations 2062, and accordingly the lower retracting member 206 may rotate in engagement with the serrations 1062 formed in the upper retracting member 106.

Here, contacting parts of the upper retracting member 106 and the lower retracting member 206 may be located in front of a knee joint. Thus, it is possible to provide a traction force to the knee retractor 10 while the upper retracting member 106 and the lower retracting member 206 rotate.

The traction force in the knee retractor 10 will be described based on a principle of pulling out a nail with a hammer with reference to FIGS. 3A and 3B.

FIG. 3A illustrates an example in which a nail is put in a hammer to pull the nail out of a surface, which may correspond to a state in which a knee is bent when a knee retractor is worn on a leg.

Here, the knee retractor may be formed so that contacting parts of an upper retracting member and a lower retracting member may be located in front of a knee joint in a state in which the knee joint is bent.

FIG. 3B illustrates an example in which a nail is pulled out by rotating a hammer, which may correspond to a state in which a knee is straightened when a knee retractor is worn on a leg.

Here, an upper retracting member and a lower retracting member of the knee retractor may rotate in engagement with each other, contacting parts of the upper retracting member and the lower retracting member may be moved, and a center of rotation of the upper retracting member and the lower retracting member may be moved toward a front side of the knee joint.

As described above, a traction force may be generated while the upper retracting member and the lower retracting member rotate in engagement with each other, and accordingly a gap between knee joints, for example, a distance between an end portion of a thigh region and an end portion of a lower leg region may be increased by the traction force. As a result, it is possible to relieve pain of the knee joint by preventing the distance between the end portion of the thigh region and the end portion of the lower leg region from being reduced when a knee is bent and straightened.

Also, a strength of a traction force generated when the upper retracting member 106 and the lower retracting member 206 are engaged may be adjusted by adjusting a position of a rotation axis of each of the upper retracting member 106 and the lower retracting member 206.

For example, when a position of a rotation axis of each of the upper retracting member 106 and the lower retracting member 206 is moved toward a front side of a knee joint, the strength of the traction force may be increased, and a distance between an end portion of a thigh region and an end portion of a lower leg region may also be increased.

In addition, a hole 2064 may be formed in the lower retracting member 206, to fix the lower retracting member 206 to the guide member 500. For example, a protrusion 524 formed in the guide member 500 may be inserted into the hole 2064.

Also, the lower frame 200 may include a fixing element 208 located at an end of the first portion 202 of the lower frame 200.

For example, the fixing element 208 may be formed integrally with or separately from the first portion 204.

A strap, in particular, a fourth strap 640 may pass through the fixing element 208, to fix the lower frame 200 to the band portion 600.

However, a shape or a structure of the fixing element 208 is not limited thereto, and the fixing element 208 may have any shape or structure to fix the lower frame 200 to the band portion 600.

The upper side frames 300 may be located on both sides of the above-described upper frame 100, and the lower side frames 400 may be located on both sides of the above-described lower frame 200.

The upper side frames 300 may be located on both sides of the thigh region.

For example, the upper side frames 300 may be located on a medial side and a lateral side of the thigh region.

The upper side frame 300 may each include an upper side straight member 302 that extends from a side of the thigh region, and an upper side diagonal member 304 that extends from the upper side straight member 302 toward the upper retracting member 106.

Here, upper side diagonal member 304 may be formed to be inclined from both sides of the thigh region toward the front side of the knee joint.

Also, a hole 3042 may be formed in an end portion of the upper side diagonal member 304.

The hole 3042 may correspond to the hole 1064 formed in the upper retracting member 106 of the upper frame 100.

Accordingly, in a state in which the hole 3042 formed in the upper side diagonal member 304 and the hole 1064 formed in the upper retracting member 106 overlap each other, the holes 3042 and 1064 may be coupled to the protrusion 5522 of the guide member 500. In other words, the upper side diagonal member 304 and the upper frame 100 may coaxially rotate.

The above shape of the upper side frame 300 may assist the upper frame 100 in performing a traction function, and the upper side frame 300 may function as a brace or a splint to allow the knee retractor 10 to be properly worn on the thigh region.

The lower side frames 400 may be located on both sides of the lower leg region.

For example, the lower side frames 400 may be located on a medial side and a lateral side of the lower leg region.

The lower side frames 400 may each include a lower side straight member 402 that extends from a side of the lower leg region, and a lower side diagonal member 404 that extends from the lower side straight member 402 toward the lower retracting member 206.

Here, lower side diagonal members 404 may be formed to be inclined from both sides of the lower leg region toward the front side of the knee joint.

Also, a hole 4042 may be formed in an end portion of the lower side diagonal member 404.

The hole 4042 may correspond to the hole 2064 formed in the lower retracting member 206 of the lower frame 200.

Accordingly, in a state in which the hole 4042 formed in the lower side diagonal member 404 and the hole 2064 formed in the lower retracting member 206 overlap each other, the holes 4042 and 2064 may be coupled to the protrusion 524 of the guide member 500. In other words, the lower side diagonal member 404 and the lower frame 200 may coaxially rotate.

The above shape of the lower side frame 400 may assist the lower frame 200 in performing a traction function, and the lower side frame 400 may function as a brace or a splint to allow the knee retractor 10 to be properly worn on the lower leg region.

Also, end portions of the upper side frame 300 and the lower side frame 400 may have circular shapes.

However, shapes of the end portions of the upper side frame 300 and the lower side frame 400 are not limited thereto, and the end portions may have sawtooth shapes so that the upper side frame 300 and the lower side frame 400 may be engaged with each other.

As described above, the upper side frame 300 and the lower side frame 400 may be fixed coaxially with the upper retracting member 106 and the lower retracting member 206, respectively.

For example, the upper side frame 300 and the lower side frame 400 may be fixed to the upper retracting member 106 and the lower retracting member 206 in the guide member 500, and the guide member 500 will be described below.

Accordingly, the upper side frame 300 and the lower side frame 400 may rotate in conjunction with rotation of the upper retracting member 106 and the lower retracting member 206, respectively.

For example, when the upper retracting member 106 rotates while a knee is straightened, the upper side frame 300 may rotate in a rotation direction of the upper retracting member 106, and the lower side frame 400 may rotate in a rotation direction of the lower retracting member 206.

Thus, similarly to the upper frame 100 and the lower frame 200, the upper side frame 300 and the lower side frame 400 may perform a function of supporting a knee joint and a function of enhancing a wearing sensation of the knee retractor 10 on a thigh region and a lower leg region at the same time.

Also, the upper frame 100, the lower frame 200, the upper side frame 300 and the lower side frame 400 may be formed of plastic, for example, polyethylene (PE) materials, to enhance a wearing sensation and reduce a weight.

However, materials of the upper frame 100, the lower frame 200, the upper side frame 300 and the lower side frame 400 are not limited to plastic materials, and any material capable of being properly bent to fit a user's body shape and returning to the original shape may be used.

Also, the upper frame 100, the lower frame 200, the upper side frame 300 and the lower side frame 400 may be fixed to the guide member 500.

In the guide member 500, a rotation axis of each of the upper frame 100, the lower frame 200, the upper side frame 300 and the lower side frame 400 may be located in the guide member 500.

The guide member 500 may extend vertically, for example, in a direction from the upper frame 100 toward the lower frame 200 or from the upper side frame 300 toward the lower side frame 400.

The guide member 500 may include a first guide element 510 located toward the lateral side of the knee joint, and a second guide element 520 located toward the knee joint to face the first guide element 510.

The first guide element 510 may include a first hole 512 and a second hole 514, and the second guide element 520 may include a first protrusion 522 and a second protrusion 524 that respectively correspond to the first hole 512 and the second hole 514.

The hole 1064 of the upper retracting member 106 and the hole 3042 of the upper side diagonal member 304 may be coupled to the first protrusion 522, and the hole 2064 of the lower retracting member 206 and the hole 4042 of the lower side diagonal member 404 may be coupled to the second protrusion 524.

Also, the first protrusion 522 may be inserted into the first hole 512, and accordingly the upper frame 100 and the upper side frame 300 may be completely fixed to the guide member 500. The second protrusion 524 may be inserted into the second hole 514, and accordingly the lower frame 200 and the lower side frame 400 may be completely fixed to the guide member 500.

The upper frame 100, in particular, the upper retracting member 106, and the upper side frame 300 may rotate together about the first protrusion 512, and the lower frame 200, in particular, the lower retracting member 206, and the lower side frame 400 may rotate together about the second protrusion 514.

Also, since the contacting parts of the upper retracting member 106 and the lower retracting member 206 are located in front of the knee joint, as described above, the guide member 500 may also be located in front of the knee joint.

Thus, the guide member 500 may function to determine a point at which a traction force is generated while fixing the upper frame 100, the lower frame 200, the upper side frame 300 and the lower side frame 400.

Here, a strength of the traction force may be adjusted by moving a position of the guide member 500. For example, as the position of the guide member 500 is moved from a side of the knee joint toward the front side, the traction force may increase.

Although not shown in the drawings, the knee retractor 10 may further include a position movement member (not shown), and the position of the guide member 500 may be moved by the position movement member to adjust the traction force.

Referring to FIGS. 4A through 4C, a shape of the knee retractor 10 may be changed while the knee joint is straightened.

Referring to FIG. 4A, in a state in which a knee is bent, a thigh region may be surrounded by the upper frame 100 and the upper side frame 300, and a lower leg region may be surrounded by the lower frame 200 and the lower side frame 400.

Here, the upper frame 100, the lower frame 200, the upper side frame 300 and the lower side frame 400 may be formed of, for example, a polyethylene material, and accordingly the shape may be changed based on a shape of the thigh region and the lower leg region.

Also, an angle between the upper frame 100 and the lower frame 200 with respect to the guide member 500 may be greater than 90 degrees and less than 180 degrees, and an angle between the upper side frame 300 and the lower side frame 400 with respect to the guide member 500 may be approximately 90 degrees.

The guide member 500 may be located in front of the knee joint, and accordingly the upper frame 100, the lower frame 200, the upper side frame 300 and the lower side frame 400 may smoothly rotate around the knee joint.

Referring to FIG. 4B, in a state in which the knee is straightened, the thigh region may be surrounded by the upper frame 100 and the upper side frame 300, and the lower leg region may be surrounded by the lower frame 200 and the lower side frame 400.

Similarly to FIG. 4A, the upper frame 100, the lower frame 200, the upper side frame 300 and the lower side frame 400 may be formed of, for example, a polyethylene material, and accordingly the shape may be changed based on a shape of the thigh region and the lower leg region.

Also, an angle between the upper frame 100 and the lower frame 200 with respect to the guide member 500 may be 180 degrees, and an angle between the upper side frame 300 and the lower side frame 400 with respect to the guide member 500 may be approximately 180 degrees.

The guide member 500 may be located in front of the knee joint, and accordingly a traction force may be provided to the knee joint, to relieve pain of the knee joint by broadening a gap between end portions of the thigh region and the lower leg region.

Referring to FIG. 4C, the upper frame 100, the lower frame 200, the upper side frame 300 and the lower side frame 400 may be formed of easily bendable materials, to be freely deformed.

Thigh regions and lower leg regions of users may have different sizes. For example, the upper frame 100 and the lower frame 200 may be deformed in a width direction of each of a thigh region and a lower leg region, and the upper side frame 300 and the lower side frame 400 may surround the thigh region and the lower leg region in a state in which the upper frame 100 and the lower frame 200 are deformed.

In an example, when a thigh region and a lower leg region of a user are large in size, the upper frame 100 and the lower frame 200 may be deformed to be more flat, and the upper side frame 300 and the lower side frame 400 may be deformed in a width direction of the thigh region and the lower leg region to be farther away from a knee joint than usual.

In another example, when a thigh region and a lower leg region of a user are small in size, the upper frame 100 and the lower frame 200 may be deformed to be more curved in a width direction of the thigh region and the lower leg region, and the upper side frame 300 and the lower side frame 400 may be deformed in the width direction of the thigh region and the lower leg region to be closer to a knee joint than usual.

In other words, the upper frame 100, the lower frame 200, the upper side frame 300 and the lower side frame 400 may be formed of deformable materials, and thus it is possible to further enhance a wearing sensation regardless of an influence of a body condition of a user.

Referring back to FIG. 1, the knee retractor 10 may further include a band portion 600 provided to surround a thigh region and a lower leg region.

The band portion 600 may be formed of, for example, air mesh or spandex material, to enhance a ventilation and a wearing sensation. In other words, sweat may be smoothly discharged through the air mesh, and the spandex material may feel smooth in a case of contact with a skin.

Thus, a user may smoothly enjoy sports even while wearing the knee retractor 10, and it is possible to prevent a skin trouble due to a direct contact with a skin.

Also, the upper frame 100, the lower frame 200, the upper side frame 300 and the lower side frame 400 may be mounted in the band portion 600, so that the upper frame 100, the lower frame 200, the upper side frame 300 and the lower side frame 400 may effectively operate.

The band portion 600 may include a plurality of straps, and accordingly the upper frame 100, the lower frame 200, the upper side frame 300 and the lower side frame 400 may be mounted in the band portion 600 using the plurality of straps.

The plurality of straps may include the first strap 610, a second strap 620, a third strap 630 and the fourth strap 640.

The first strap 610 may pass through the upper frame 100, in particular, the fixing element 108, and may fix the upper frame 100 to the band portion 600.

The second strap 620 may be spaced downwardly from the first strap 610 and may fix the upper side frame 300 to the band portion 600.

The third strap 630 may be spaced downwardly from the second strap 620 and may fix the lower side frame 400 to the band portion 600.

The fourth strap 640 may be spaced downwardly from the third strap 630 and may pass through the lower frame 200, in particular, the fixing element 208, to fix the lower frame 200 to the band portion 600.

The first strap 610, the second strap 620, the third strap 630 and the fourth strap 640 may function to securely fix the knee retractor 10 and a user's leg.

Also, the first strap 610, the second strap 620, the third strap 630 and the fourth strap 640 may be easily attached to or detected from the band portion 600 using a Velcro.

For example, when a user wears the band portion 600 of the knee retractor 10 on a leg, the fourth strap 640, the third strap 630, the second strap 620 and the first strap 610 may be sequentially tightened, to allow the band portion 600 to come into close contact with a thigh region and a lower leg region and to press the leg.

As described above, a knee retractor according to an example embodiment may be worn on a patient's leg by a band portion, to effectively operate, to press the leg, and to securely fix the leg by utilizing a plurality of straps.

The knee retractor has been described above, and another example of a knee retractor will be described below.

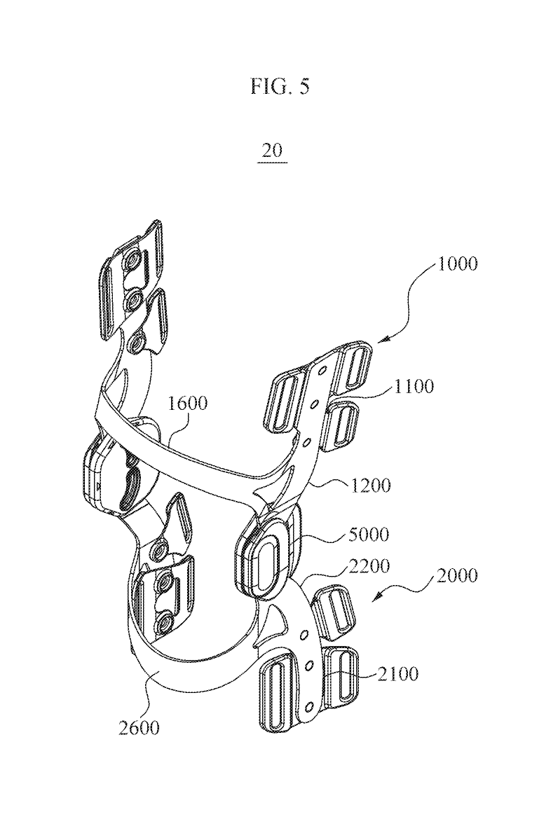

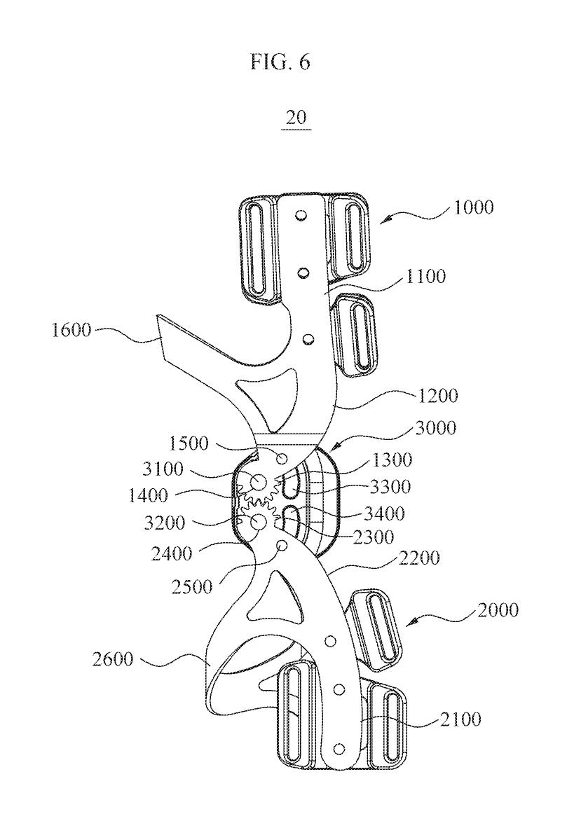

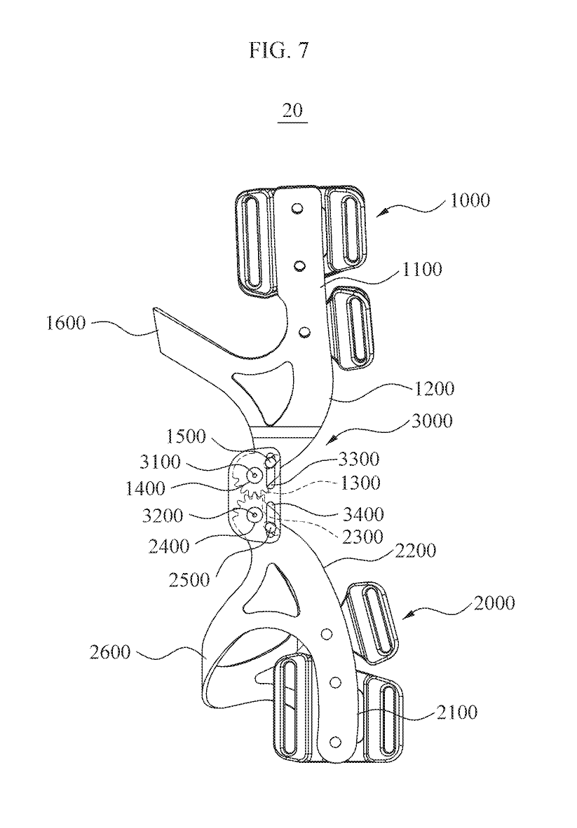

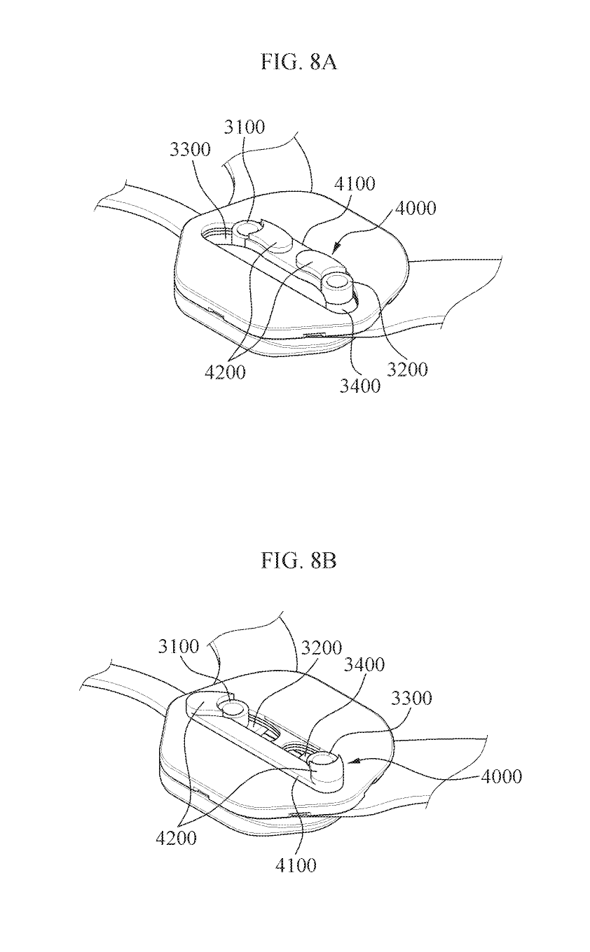

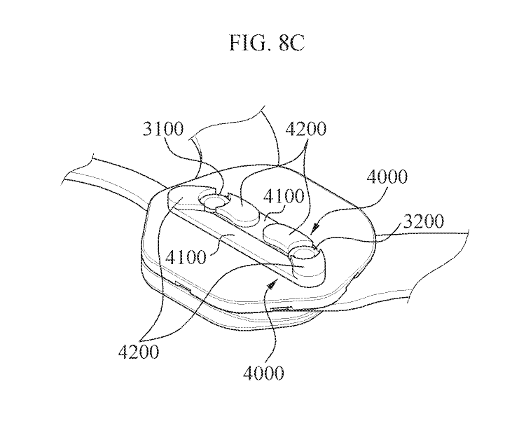

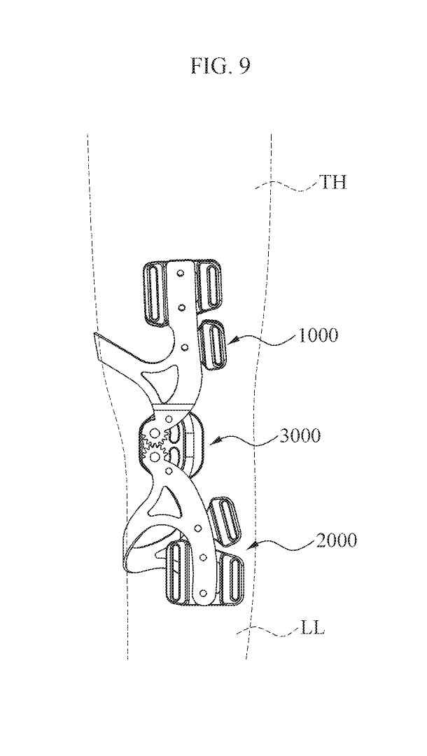

FIG. 5 is a perspective diagram of a knee retractor according to an example embodiment, FIG. 6 illustrates an example in which a cover is removed from the knee retractor and guide grooves have curved shapes, and FIG. 7 illustrates an example in which a cover is removed from the knee retractor and guide grooves have linear shapes. FIGS. 8A through 8C illustrate examples of a stopper mounted in the knee retractor, FIG. 9 illustrates an example in which a knee joint is straightened when the knee retractor is worn, and FIG. 10 illustrates an example in which a knee joint is bent when the knee retractor is worn.

Referring to FIG. 5, a knee retractor 20 according to an example embodiment may include an upper frame 1000 and a lower frame 2000.

The upper frame 1000 may be provided to be worn on a thigh region (for example, the thigh region TH of FIG. 9 or 10).

The upper frame 1000 may include an upper straight member 1100 that extends from a side of the thigh region, and an upper diagonal member 1200 that connects the upper straight member 1100 and an upper retracting member 1300.

The lower frame 2000 may be provided to be worn on a lower leg region (for example, a lower leg region LL of FIG. 9 or 10).

The lower frame 2000 may include a lower straight member 2100 that extends from a side of the thigh region, and a lower diagonal member 2200 that connects the lower straight member 2100 and a lower retracting member 2300.

Referring to FIG. 6, the upper retracting member 1300 and the lower retracting member 2300 may be located adjacent to a side of a knee joint at an end of the upper frame 1000 and an end of the lower frame 2000, respectively.

The upper retracting member 1300 and the lower retracting member 2300 may be formed integrally with the upper frame 1000 and the lower frame 2000, or formed separately from the upper frame 1000 and the lower frame 2000.

Also, the upper retracting member 1300 and the lower retracting member 2300 may each have a sawtooth shape.

A portion of each of the upper retracting member 1300 and the lower retracting member 2300 has a sawtooth shape as shown in the drawing, however, it is obvious that a circumferential edge of each of the upper traction member 1300 and the lower traction member 2300 may have a sawtooth shape.

In particular, the upper retracting member 1300 and the lower retracting member 2300 may each have a shape of a circle including a quadrant.

For example, when the upper retracting member 1300 and the lower retracting member 2300 have quadrants, the quadrants may be located so that an arc of each of the quadrants may come in contact with each other.

In this example, contacting parts of the upper retracting member 1300 and the lower retracting member 2300 may be located in front of the knee joint.

Accordingly, a center of rotation of the upper retracting member 1300 and the lower retracting member 2300 may be located in front of the knee joint, and the knee joint may be supported so that a user may naturally straighten and bend the knee joint.

Also, based on a position of a contacting part of the upper retracting member 1300 and the lower retracting member 2300, a shape of each of the upper diagonal member 1200 and the lower diagonal member 2200 may change.

In an example, the upper diagonal member 1200 and the lower diagonal member 2200 may be curved from the upper straight member 1100 and the lower straight member 2100 toward the upper retracting member 1300 and the lower retracting member 2300. In another example, it is obvious that the upper diagonal member 1200 and the lower diagonal member 2200 may extend from the upper straight member 1100 and the lower straight member 2100 instead of being inclined.

The upper retracting member 1300 and the lower retracting member 2300 formed as described above may rotate in engagement with each other. Also, an angle between the upper frame 1000 and the lower frame 2000 may change by a rotation direction of each of the upper retracting member 1300 and the lower retracting member 2300.

The upper retracting member 1300 and the lower retracting member 2300 may be symmetrical to each other, and may rotate in opposite directions.

In an example, when a knee joint is straightened, the upper retracting member 1300 may rotate counterclockwise, or the lower retracting member 2300 may rotate clockwise. Accordingly, the angle between the upper frame 1000 and the lower frame 2000 may increase.

In another example, when the knee joint is bent, the upper retracting member 1300 may rotate clockwise, or the lower retracting member 2300 may rotate counterclockwise. Accordingly, the angle between the upper frame 1000 and the lower frame 2000 may decrease.

The knee retractor 20 may further include a guide member 3000 in which a rotation axis of each of the upper retracting member 1300 and the lower retracting member 2300 is located.

The guide member 3000 may include a first protrusion 3100, a second protrusion 3200, a first guide groove 3300 and a second guide groove 3400.

The first protrusion 3100 may be located in a center of rotation, that is, a rotation axis of the upper retracting member 1300, and the second protrusion 3200 may be located in a center of rotation, that is, a rotation axis of the lower retracting member 2300.

The upper retracting member 1300 may include an upper groove 1400 capable of being coupled to the first protrusion 3100 of the guide member 3000, and the lower retracting member 2300 may include a lower groove 2400 capable of being coupled to the second protrusion 3200 of the guide member 3000.

The first protrusion 3100, the second protrusion 3200, the upper groove 1400 and the lower groove 2400 may be located so that the upper retracting member 1300 and the lower retracting member 2300 may rotate in mesh.

Also, since the contacting parts of the upper retracting member 1300 and the lower retracting member 2300 are located in front of the knee joint, the first protrusion 3100 and the second protrusion 3200 of the guide member 3000 may also be located in front of the knee joint.

The first guide groove 3300 and the second guide groove 3400 may allow the upper retracting member 1300 and the lower retracting member 2300 to smoothly rotate, and accordingly the first guide groove 3300 and the second guide groove 3400 may be located further behind the knee joint than the upper retracting member 1300 and the lower retracting member 2300.

An upper protruding element 1500 may be inserted into the first guide groove 3300 and may be guided, and a lower protruding element 2500 may be inserted into the second guide groove 3400 and may be guided.

The upper protruding element 1500 may protrude inward from the upper frame 1000, and the lower protruding element 2500 may protrude inward from the lower frame 2000.

For example, the upper protruding element 1500 and the lower protruding element 2500 may move inward from upper diagonal member 1200 and the lower diagonal member 2200, respectively.

In this example, the first guide groove 3300 and the second guide groove 3400 may be formed along a moving route of the upper protruding element 1500 and a moving route of the lower protruding element 2500, respectively. Also, the upper protruding element 1500 and the lower protruding element 2500 may be located to be inserted into first guide groove 3300 and the second guide groove 3400, respectively.

The first guide groove 3300 and the second guide groove 3400 may be symmetrical to each other, and may each have a curved shape. The upper protruding element 1500 and the lower protruding element 2500 may also be symmetrical to each other, and may move while drawing curved tracks.

As shown in FIG. 7, the first guide groove 3300 and the second guide groove 3400 may be symmetrical to each other, and may each have a linear shape. The upper protruding element 1500 and the lower protruding element 2500 may also be symmetrical to each other, and may move while drawing linear tracks within the first guide groove 3300 and the second guide groove 3400.

Here, the upper protruding element 1500 and the lower protruding element 2500 may extend outwardly as well as inwardly from the upper diagonal member 1200 and the lower diagonal member 2200, respectively.

In an example, when a knee joint is straightened, the angle between the upper frame 1000 and the lower frame 2000 may increase, and the upper retracting member 1300 and the lower retracting member 2300 may rotate based on an increase in the angle. Accordingly, the upper protruding element 1500 and the lower protruding element 2500 may be guided toward an opposite end of the upper frame 1000 and an opposite end of the lower frame 2000 within the first guide groove 3300 and the second guide groove 3400, respectively, and a distance between the upper protruding element 1500 and the lower protruding element 2500 may gradually increase.

In another example, when the knee joint is bent, the angle between the upper frame 1000 and the lower frame 2000 may decrease, and the upper retracting member 1300 and the lower retracting member 2300 may rotate based on a decrease in the angle. Accordingly, the upper protruding element 1500 and the lower protruding element 2500 may be guided toward the end of the upper frame 1000 and the end of the lower frame 2000 within the first guide groove 3300 and the second guide groove 3400, respectively, and the distance between the upper protruding element 1500 and the lower protruding element 2500 may gradually decrease.

By the guide member 3000 formed as described above, the upper retracting member 1300 and the lower retracting member 2300 may smoothly rotate, and the knee joint may be naturally straightened and bent.

Referring to FIGS. 8A, 8B and 8C, a stopper 4000 may be mounted in the first guide groove 3300 and the second guide groove 3400.

The stopper 4000 may include a straight portion 4100, and pivoting portions 4200 that are located in both ends of the straight portion 4100.

The straight portion 4100 may be located outside the first guide groove 3300 and the second guide groove 3400, and the pivoting portions 4200 may be located in the first guide groove 3300 and the second guide groove 3400, respectively.

The pivoting portions 4200 may be pivotable with respect to the straight portion 4100. Accordingly, an angle between the straight portion 4100 and the pivoting portions 4200 may be adjusted.

In an example, when a long straight portion 4100 is provided as shown in FIG. 8A, pivoting portions 4200 may be mounted far away from each other in the first guide groove 3300 and the second guide groove 3400. Thus, it is possible to prevent the angle between upper frame 1000 and the lower frame 2000 from further increasing, by preventing the upper protruding element 1500 and the lower protruding element 2500 from moving far away from each other.

In another example, when a short straight portion 4100 is provided as shown in FIG. 8B, pivoting portions 4200 may be installed close to each other in the first guide groove 3300 and the second guide groove 3400. Thus, it is possible to prevent the angle between the upper frame 1000 and the lower frame 2000 from further decreasing, by preventing the upper protruding element 1500 and the lower protruding element 2500 from moving close to each other.

Although the stoppers 4000 having different shapes are individually mounted as shown in FIGS. 8A and 8B, the stoppers 4000 with different shapes may be mounted together as shown in FIG. 8C. For example, the upper protruding element 1500 and the lower protruding element 2500 may be fixed by the stopper 4000 within the first guide groove 3300 and the second guide groove 3400, and accordingly may not move in any direction.

Also, to facilitate installation of the stopper 4000, the guide member 3000 may have a hollow portion in a position in which the stopper 4000 is to be mounted.

Thus, the stopper 4000 may limit movements of the upper protruding element 1500 and the lower protruding element 2500 within the first guide groove 3300 and the second guide groove 3400, may limit rotation of the upper retracting member 1300 and the lower retracting member 2300, and may fix the angle between the upper frame 1000 and the lower frame 2000.

The upper frame 1000 and the lower frame 2000 may include an upper connection member 1600 and a lower connection member 2600.

The upper connection member 1600 may connect upper straight members 1100 extending from both sides of the thigh region. The lower connection member 2600 may connect lower straight members 2100 extending from both sides of the lower leg region.

Here, the upper connection member 1600 and the lower connection member 2600 may be formed between the opposite end of the upper frame 1000 and the upper retracting member 1300, and between the opposite end of the lower frame 2000 and the lower retracting member 2300, respectively.

For example, the upper connection member 1600 and the lower connection member 2600 may be located adjacent to the knee joint, and the upper connection member 1600 and the lower connection member 2600 may be shaped to surround the knee joint.

The upper connection member 1600 may be curved to surround an upper portion of the knee joint, and the lower connection member 2600 may be curved to surround a lower portion of the knee joint.

Due to the upper connection member 1600 and the lower connection member 2600 formed adjacent to the knee joint, a material with a relatively low strength may be used to manufacture the upper frame 1000 and the lower frame 2000. Thus, it is possible to reduce manufacturing costs.

In other words, when the upper connection member 1600 is formed in a middle portion of the upper straight member 1100 of the upper frame 1000 and when the lower connection member 2600 is formed in a middle portion of the lower straight member 2100 of the lower frame 2000, the upper frame 1000 and the lower frame 2000 may be formed of a material or elastic material with a low strength, in comparison to when the upper connection member 1600 and the lower connection member 2600 are formed in the opposite end of the upper frame 1000 and the opposite end of the lower frame 2000, respectively.

When the upper straight member 1100 and the lower straight member 2100 are formed of elastic materials in the upper frame 1000 and the lower frame 2000, the knee retractor 20 may be used regardless of sizes of thigh regions and lower leg regions of users, and a wearing sensation may be further enhanced.

Also, the knee retractor 20 may include a cover 5000 configured to protect the upper retracting member 1300, the lower retracting member 2300 and the guide member 3000.

The cover 5000 may prevent the upper retracting member 1300, the lower retracting member 2300 and the guide member 3000 from being damaged by an outside exposure, and accordingly a life of the knee retractor 20 may be extended.

Also, the cover 5000 may be detachably provided, and accordingly it is possible to facilitate maintenance of the upper retracting member 1300, the lower retracting member 2300 and the guide member 3000.

The knee retractor configured as described above may be worn on a knee and may operate as follows.

In an example, when a knee joint in a bent position is straightened, as shown in FIGS. 6 and 9, the upper retracting member 1300 may rotate counterclockwise about the first protrusion 3100 of the guide member 3000, or the lower retracting member 2300 may rotate clockwise about the second protrusion 3200 of the guide member 3000.

In this example, the first protrusion 3100 may be inserted into the upper groove 1400 in the upper retracting member 1300, and the second protrusion 3200 may be inserted into the lower groove 2400 in the lower retracting member 2300.

Accordingly, the upper protruding element 1500 in the upper frame 1000 may be moved toward the opposite end of the upper frame 1000 along the first guide groove 3300 of the guide member 3000, or the lower protruding element 2500 in the lower frame 2000 may be moved toward the opposite end of the lower frame 2000 along the second guide groove 3400 of the guide member 3000.

The upper retracting member 1300 and the lower retracting member 2300 may rotate so that the angle between the upper frame 1000 and the lower frame 2000 may increase.

Referring to FIGS. 8 and 9, when rotations of the upper retracting member 1300 and the lower retracting member 2300 are terminated, the stopper 4000 may be mounted in the guide member 3000 to fix the above state.

In another example, when a knee joint in a straightened position is bent, as shown in FIGS. 6 and 10, the upper retracting member 1300 may rotate clockwise about the first protrusion 3100 of the guide member 3000, or the lower retracting member 2300 may rotate counterclockwise about the second protrusion 3200 of the guide member 3000.

Accordingly, the upper protruding element 1500 in the upper frame 1000 may be moved toward the end of the upper frame 1000 along the first guide groove 3300 of the guide member 3000, or the lower protruding element 2500 in the lower frame 2000 may be moved toward the end of the lower frame 2000 along the second guide groove 3400 of the guide member 3000.

The upper retracting member 1300 and the lower retracting member 2300 may rotate so that the angle between the upper frame 1000 and the lower frame 2000 may increase.

Referring to FIGS. 8 and 10, when rotations of the upper retracting member 1300 and the lower retracting member 2300 are terminated, the stopper 4000 may be mounted in the guide member 3000 to fix the above state.

The knee retractor configured as described above may allow a rotation axis of each of an upper retracting member and a lower retracting member to be located in front of a knee joint, and may efficiently limit, using a stopper, an angle at which the knee joint is bent. Also, an upper connection member and a lower connection member may be located adjacent to the knee joint, and accordingly it is possible to reduce manufacturing costs and facilitate manufacturing. Furthermore, the knee retractor may protect cartilages by reducing load applied to the knee joint, may induce healing of the knee joint by relieving pain of the knee joint, and may support the knee joint so that a user may naturally straighten and bend the knee joint.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A knee retractor 10 comprising:
an upper frame 100 located on a thigh region and comprising an upper retracting member 106 located at an end;
a lower frame 200 located on a lower leg region and comprising a lower retracting member 206 that is located at an end and that is rotatable in engagement with the upper retracting member 106; and
a band portion 600 provided to surround the thigh region and the lower leg region, the upper frame 100 and the lower frame 200 being fixed to the band portion 600,
wherein contacting parts of the upper retracting member 106 and the lower retracting member 206 are located in front of a knee joint.

2. The knee retractor 10 of claim 1, wherein an end portion of each of the upper retracting member 106 and the lower retracting member 206 has serrations 1062 so that the upper retracting member 106 and the lower retracting member 206 rotate in engagement with each other.

3. The knee retractor 10 of claim 1, wherein
each of the upper frame 100 and the lower frame 200 has a “Y” shape, and
the upper frame 100 and the lower frame 200 comprise:
first portions 102 and 202 extending from the knee joint in a longitudinal direction of each of the thigh region and the lower leg region; and
second portions 104 and 204 connected to the first portions 102 and 202 and provided to surround an upper portion and a lower portion of the knee joint.

4. The knee retractor 10 of claim 1, further comprising:
upper side frames 300 located on both sides of the thigh region, and each having one end fixed coaxially with the upper retracting member 106; and
lower side frames 400 located on both sides of the lower leg region, and each having one end fixed coaxially with the lower retracting member 206.

5. The knee retractor 10 of claim 4, wherein
the upper side frames 300 each comprise an upper side straight member 302 extending from a side of the thigh region, and an upper side diagonal member 304 extending from the upper side straight member 302 toward the upper retracting member 106, and
the lower side frames 400 each comprise a lower side straight member 402 extending from a side of the lower leg region, and a lower side diagonal member 404 extending from the lower side straight member 402 toward the lower retracting member 206.

6. The knee retractor 10 of claim 4, wherein the upper side frame 300 rotates in conjunction with a rotation of the upper retracting member 106, and the lower side frame 400 rotates in conjunction with a rotation of the lower retracting member 206.

7. The knee retractor 10 of claim 4, wherein
the band portion 600 comprises a plurality of straps, and
the plurality of straps comprise:
a first strap 610 passing through the upper frame 100 and configured to fix the upper frame 100 to the band portion 600;
a second strap 620 spaced downwardly from the first strap 610 and configured to fix the upper side frame 300 to the band portion 600;
a third strap 630 spaced downwardly from the second strap 610 and configured to fix the lower side frame 400 to the band portion 600; and
a fourth strap 640 spaced downwardly from the third strap 630, passing through the lower frame 200 and configured to fix the lower frame 200 to the band portion 600.

8. The knee retractor 10 of claim 4, further comprising:
a guide member 500 in which a rotation axis of each of the upper retracting member 106, the lower retracting member 206, the upper side frame 300 and the lower side frame 400 is located.

9. A knee retractor 10 comprising:
an upper frame 100 located on a thigh region and comprising an upper retracting member 106 located at an end;
a lower frame 200 located on a lower leg region and comprising a lower retracting member 206 that is located at an end and that is rotatable in engagement with the upper retracting member 106;
upper side frames 300 located on both sides of the thigh region, and each having one end fixed coaxially with the upper retracting member 106; and
lower side frames 400 located on both sides of the lower leg region, and each having one end fixed coaxially with the lower retracting member 206,
wherein the upper side frame 300 rotates in conjunction with a rotation of the upper retracting member 106, and the lower side frame 400 rotates in conjunction with a rotation of the lower retracting member 206.

10. The knee retractor 10 of claim 9, wherein contacting parts of the upper retracting member 106 and the lower retracting member 206 are located in front of a knee joint.

11. The knee retractor 10 of claim 10, wherein
the upper side frames 300 each comprise an upper side straight member 302 extending from a side of the thigh region, and an upper side diagonal member 304 extending from the upper side straight member 302 toward the contacting parts of the upper retracting member 106 and the lower retracting member 206, and
the lower side frames 400 each comprise a lower side straight member 402 extending from a side of the lower leg region, and a lower side diagonal member 404 extending from the lower side straight member 402 toward the contacting parts of the upper retracting member 106 and the lower retracting member 206.

12. The knee retractor 10 of claim 9, further comprising:
a band portion 600 provided to surround the thigh region and the lower leg region,
wherein the upper frame 100, the lower frame 200, the upper side frame 300 and the lower side frame 400 are fixed to the band portion 600.

13. The knee retractor 10 of claim 9, further comprising:
a guide member 500 in which a rotation axis of each of the upper retracting member 106, the lower retracting member 206, the upper side frame 300 and the lower side frame 400 is located.

14. A knee retractor 10 comprising:
an upper side frame 300 comprising an upper side straight member 302 extending straightly from a side of a thigh region, and an upper side diagonal member 304 extending diagonally from the upper side straight member 302 toward an upper retracting member 106; and
a lower side frame 400 comprising a lower side straight member 402 extending straightly from a side of a lower leg region, and a lower side diagonal member 404 extending diagonally from the lower side straight member 402 toward a lower retracting member 206,
wherein a strength of a traction force is adjusted by adjusting a position of a rotation axis of each of the upper retracting member 106 and the lower retracting member 206.

15. The knee retractor 10 of claim 14, wherein the strength of the traction force increases as the position of the rotation axis of each of the upper frame 100 and the lower frame 200 is moved toward a front side of a knee joint, and a distance between an end portion of the thigh region and an end portion of the lower leg region is increased by the traction force.

16. The knee retractor 10 of claim 14, wherein
the upper retracting member 106 and the lower retracting member 206 have circular shapes or quadrant shapes, and
an end portion of each of the upper retracting member 106 and the lower retracting member 206 has serrations 1062 so that the upper retracting member 106 and the lower retracting member 206 rotate in engagement with each other.

17. The knee retractor 10 of claim 14, further comprising:
a guide member 500 in which a rotation axis of each of the upper retracting member 106, the lower retracting member 206, the upper side frame 300 and the lower side frame 400 is located,
wherein the guide member 500 is formed to extend from the upper side frame 300 toward the lower side frame 400.

18. The knee retractor 10 of claim 17, wherein
the guide member 500 comprises:
a first guide element 510 located toward a lateral side of the knee joint; and
a second guide element 520 located toward the knee joint to face the first guide element 510,
the first guide element 510 comprises a first hole 512 and a second hole 514 that are spaced apart from each other, and
the second guide element 520 comprises a first protrusion 522 corresponding to the first hole 512 and a second protrusion 524 corresponding to the second hole 514.

19. The knee retractor 10 of claim 18, wherein
a hole 1064 is formed in a central portion of the upper retracting member 106,
a hole 3042 is formed adjacent to an end portion of the upper side frame 300, and
the hole 1064 of the upper retracting member 106 and the hole 3042 of the upper side frame 300 are coupled to the first protrusion 522 to overlap each other, and the upper frame 100 and the upper side frame 300 rotate together about the first protrusion 522.

20. The knee retractor 10 of claim 18, wherein
a hole 2064 is formed in a central portion of the lower retracting member 206,
a hole 4042 is formed adjacent to an end portion of the lower side frame 400, the hole 2064 of the lower retracting member 206 and the hole 4042 of the lower side frame 400 are coupled to the second protrusion 524 to overlap each other, and the lower frame 200 and the lower side frame 400 rotate together about the second protrusion 524.
